(19) United States
(12) Patent Application Publication
(10) Pub. No.: US 2008/0146891 A1
(43) Pub. Date: Jun. 19, 2008

(54) Method of monitoring physiological status and apparatus thereof

(75) Inventors: Wei-Ru Wang, Tainan City (TW); Yu-Chia Hsu, Chiayi City (TW); Shin-Mu Tseng, Tainan City (TW)

Correspondence Address: RABIN & Berdo, PC, 1101 14TH STREET, NW, SUITE 500, WASHINGTON, DC, 20005, US

(73) Assignee: INSTITUTE FOR INFORMATION INDUSTRY, Taipei City (TW)

(21) Appl. No.: 11/812,425

(22) Filed: Jun. 19, 2007

(30) Foreign Application Priority Data
Dec. 13, 2006 (TW) 095146765

(51) Int. Cl. A61B 5/00

(52) U.S. Cl. 600/300

(57) ABSTRACT

A method of monitoring physiological status is periodically inspecting physiological status of a user, obtaining a physiological signal value, and determining whether the physiological signal value is normal or not. The method includes collecting a plurality of specific physiological values, calculating a reference value and a reference range according to the specific physiological values, and determining and displaying whether the physiological signal value of the specific section of the next inspection period falls within the reference value and the reference range according to the reference value and the reference range. The update of the above reference value and reference range is determined when a next physiological value is collected, so as to maintain the accuracy of the reference value and reference range. A care device of monitoring physiological status is also included in the embodiment.

This non-provisional application claims priority under 35 U.S.C. § 119(a) on Patent Application No(s). 095146765 filed in Taiwan, R.O.C. on Dec. 13, 2006, the entire contents of which are hereby incorporated by reference.

### BACKGROUND OF THE INVENTION

1. Field of Invention

The present invention relates to a physiological signal comparison method, and more particularly to a method of adjusting the reference value according to the actual physiological status of a user, so as to accurately determine whether the physiological signal value obtained in a next inspection period is normal or not.

2. Related Art

A common physiological signal inspection device usually includes an inspection unit, a microprocessor, and a display unit, wherein the microprocessor is electrically connected to the display unit and the inspection unit.

The inspection unit is generally used to measure at least one physiological signal of a human body, and the measured condition signal is analyzed by the microprocessor to be converted into a digital physiological signal value. Afterwards, the microprocessor outputs the analyzed physiological signal value to the display unit for display, such that a user can acquire the current physiological status.

Another circumstance is to inspect long-term changes in the physiological status of a user. To achieve the purpose, a recording module is disposed in the inspection device. After measuring a physiological signal, the inspection unit transmits the physiological signal to the microprocessor. Next, the microprocessor converts the physiological signal into a digital physiological signal value, then outputs the physiological signal value to the display unit, and meanwhile stores the physiological signal value into the recording unit to form a physiological record. The microprocessor outputs the physiological record to the display unit, such that the user can know the changes in the physiological status during the inspection period.

However, a user instead of a professional in medicine cannot understand the meaning of the physiological signal value or physiological record output or displayed by the above inspection device and cannot determine whether the displayed physiological signal value and physiological record are abnormal or not. As a result, the user cannot clearly know whether he/she is in a good physiological status.

Moreover, even if a parameter database is disposed in the inspection device to acquire the comparison information when the physiological signal value is converted from the microprocessor, thereby determining the physiological signal measured by the inspection unit is abnormal or not. As the physiological status of each user varies due to different living environment, the difference between the inspected physiological signal value and the physiological record is quite obvious. In addition, the data in the parameter database are usually stored in advance and cannot be altered. If a constant parameter is used to compare the physiological signal value and the physiological record, a false determination on the physiological status is easily made.

Further, even if the inspection module has a manipulation interface for the user to perform data alteration on the parameter database, the value is usually altered and designed by medical professionals based on the physiological status of each user. The user cannot alter the value unless he/she knows his/her own physiological status and is familiar to the manipulation interface as well as the meaning of the data in the parameter database, which is very inconvenient in use.

### SUMMARY OF THE INVENTION

Accordingly, in order to solve the above problems, the present invention provides a method of monitoring physiological status, which is used to output a related warning signal in addition to an inspection result according to the inspection result when the inspection of the physiological signal is completed, so as to inform the user whether his/her physiological status are abnormal or not. Moreover, the reference value corresponding to the inspection physiological signal is properly adjusted based on the inspection result automatically in accordance with the actual physiological status of the user, and thus whether the physiological signal value obtained in the next inspection period is abnormal or not can be determined more accurately.

The present invention provides a method of monitoring physiological status, which includes periodically inspecting physiological status of a user, obtaining a physiological signal value, and determining whether the physiological signal value is normal or not.

First, a plurality of physiological signal values is collected in a plurality of inspection periods, and the inspection periods are converted into a plurality of specific sections. In the plurality of inspection periods, all the physiological signal values obtained in the same specific section constitute a specific physiological value, and the maximum quantity of the specific physiological values may not exceed the quantity of the specific sections.

A reference value and a reference range are calculated based on the specific physiological values.

Afterwards, in the next inspection period, a physiological signal value of the specific section is obtained, and whether the physiological signal value falls within the reference value and the reference range is determined according to the reference value and the reference range.

Finally, whether the physiological signal value falls within the reference value and the reference range is displayed according to a comparison result.

The present invention provides a care device of monitoring physiological status. The care device is connected to an inspection device, and the inspection device provides at least one physiological signal value. The care device periodically inspects the physiological status of a user, obtains a physiological signal value, and determines whether the physiological signal value is normal or not.

The care device comprises a physiological database, a reference database, a receiving module, and a data processing module.

The data processing module is electrically connected to the physiological database, the reference database, and the receiving module, and the receiving module is further electrically connected to the physiological database.

The receiving module receives and stores the physiological signal values provided by the inspection device into the physiological database.

The data processing module retrieves the physiological signal values from the physiological database, calculates a reference value and a reference range according to the specific physiological values, and stores the specific physiological values in the reference database.

When the data processing module receives a physiological signal value of the specific section of the next inspection period, a corresponding reference value and reference range are retrieved from the reference database, so as to determine whether the received physiological signal value falls within the reference value and the reference range, and determine whether or not to update the specific physiological values stored in the reference database based on the comparison result.

Moreover, It is determined whether the data processing module sends out a suggestion signal according to the comparison result, so as to prompt the user or persons nearby to solve the problem when an abnormal status of the user occurs, thus quickly settling the abnormal physiological problem of the user.

The care device provided by the present invention further comprises a display unit which is a liquid crystal panel. The display unit is electrically connected to the data processing module for displaying whether the physiological signal value of the next inspection period falls within the reference value and the reference range and meanwhile displaying the information contained in the prompt signal.

The care device provided by the present invention further comprises a sound playing unit. The data processing module outputs an auxiliary sound signal to the sound playing unit when the comparison result is generated, so that the sound playing unit plays a sound corresponding to the auxiliary sound signal.

The care device provided by the present invention further comprises a signal transmission module. The signal transmission module is electrically connected to the data processing module and the receiving module, and is connected to at least one network device, such that the network device is electrically connected to the data processing module and the receiving module via the signal transmission module, thus obtaining various values of the reference value and reference range of the physiological signal value or the specific physiological value.

The techniques described herein may be implemented in hardware, software, firmware, or any combination thereof. If implemented in software, the techniques may be directed to a readable medium comprising program code, that when executed, performs one or more of the techniques described herein.

The present invention has an efficacy that cannot be achieved by the prior art. That is, after the comparison of the physiological signal value is completed according to the method and device of the present invention, the specific physiological values for comparison are automatically properly adjusted based on the comparison result, so as to accurately determine whether the physiological signal value obtained from the next inspection period is abnormal or not according to the current physiological status of the user. Moreover, a prompt signal is sent to the user based on the comparison result, such that the user can know his/her physiological status and can take actions when the abnormal physiological status occurs.

In addition, the method provided by the present invention records the physiological signals of the past, which is convenient for the user to know long-term changes of his/her physiological status, such that a recessive condition change of a certain disease can be inspected.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given herein below for illustration only, and thus are not limitative of the present invention, and wherein:

FIG. 1 is a schematic structural view of a device of the present invention;

FIG. 2A is a flow chart of a method of the present invention;

FIG. 2B is a view of the collection of the physiological signal values of the present invention;

FIG. 2C is a cut view of the time range of the present invention;

FIG. 2D is a schematic view of the inspection periods corresponding to the same time axis according to the present invention;

FIG. 2E is a schematic view of a specific section corresponding to the physiological signal values according to the present invention;

FIG. 2F is a curve diagram formed by specific physiological values according to the present invention;

FIG. 2G is a schematic comparison view of the physiological signal values and the specific physiological values according to the present invention;

FIG. 2H is a flow chart of the comparison rule according to the present invention; and

FIG. 2I is an output signal table of the data processing module according to the present invention.

### DETAILED DESCRIPTION OF THE INVENTION

The objectives, structural features, and functions of the present invention will be illustrated in detail below accompanied with the embodiments and drawings.

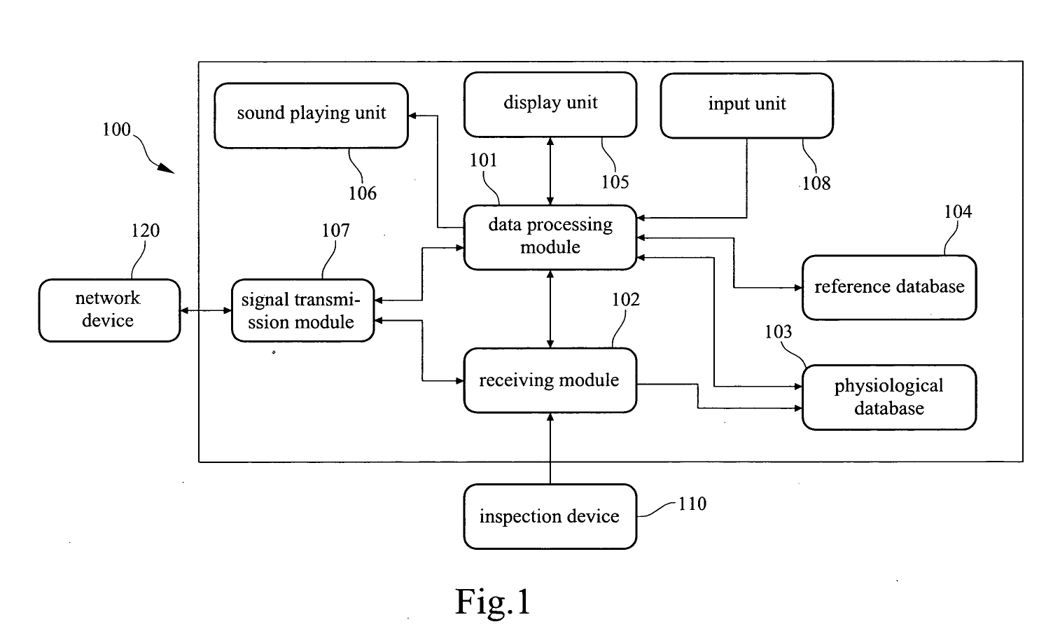

Referring to FIG. 1, it is a schematic structural view of a device of the present invention. The care device 100 includes a physiological database 103, a reference database 104, a receiving module 102, a data processing module 101, a display unit 105, a sound playing unit 106, and a signal transmission module 107. The care device 100 is externally connected to a network device 120 and an inspection device 110, wherein the inspection device 110 provides at least one physiological signal value.

The data processing module 101 is electrically connected to the physiological database 103, the reference database 104, the receiving module 102, the data processing module 101, the display unit 105, the sound playing unit 106, and the signal transmission module 107. The signal transmission module 107 is further electrically connected to the receiving module 102.

The receiving module 102 is connected to at least one external inspection device 110 for receiving and storing a plurality of physiological signal values provided by the inspection device 110 into the physiological database 103.

The data processing module 101 retrieves the physiological signal values in the same specific section from the physiological database 103 according to a plurality of inspection periods corresponding to the physiological signal values, and the retrieved physiological signal values constitute a plurality of specific physiological values, wherein a specific section is corresponding to a specific physiological value. Next, the data processing module 101 calculates a reference value and a reference range corresponding to each specific physiological value, and stores the specific physiological values into the reference database 104.

When the data processing module 101 receives a physiological signal value of a specific section of the next period, the specific physiological values corresponding to the physiological signal value are retrieved from the reference database 104, and it is determined whether the physiological signal value falls within the reference value and the reference range according to the reference value and the reference range of the specific physiological values. After that, the determination result is output by the data processing module 101, and whether the physiological signal value falls within the reference value and the reference range is displayed by the display unit 105. The data processing module 101 outputs an auxiliary sound signal and a prompt signal when the comparison result is generated, and determines whether or not to update the specific physiological values stored in the reference database 104.

The display unit 105 is a liquid crystal panel for displaying whether the physiological signal value falls within the reference value and the reference range, and displaying the content of the suggestion signal.

The sound playing unit 106 receives the auxiliary sound signal to play a sound corresponding to the auxiliary sound signal.

The signal transmission module 107 is electrically connected to the data processing module 101 and the receiving module 102, and is connected to at least one external network device 120, such that the network device 120 is electrically connected to the data processing module 101 and the receiving module 102 via the signal transmission module 107.

The network device 120 exchanges signal with the data processing module 101 and the receiving module 102 via the signal transmission module 107, so as to obtain the reference value and the reference range of the physiological signal value or the specific physiological values. Moreover, the network device 120 alters the parameter values contained in the data processing module 101 via the signal transmission module 107.

Further, the care device 100 includes an input unit 108 which is electrically connected to the data processing module 101 for the user to alter the parameter values in the data processing module 101.

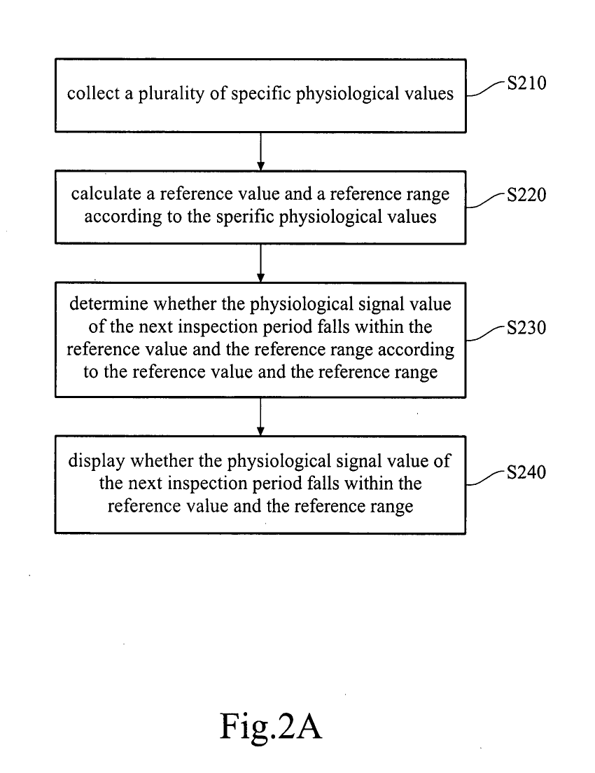

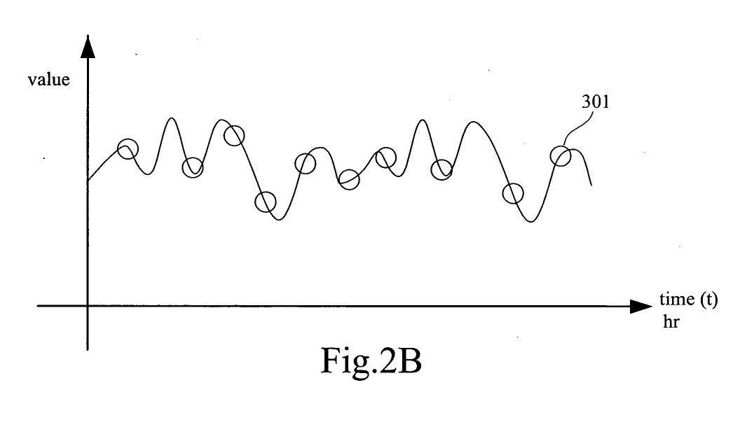

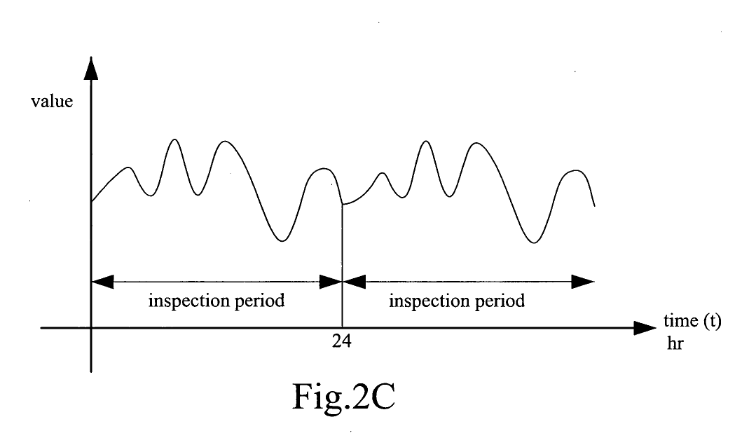

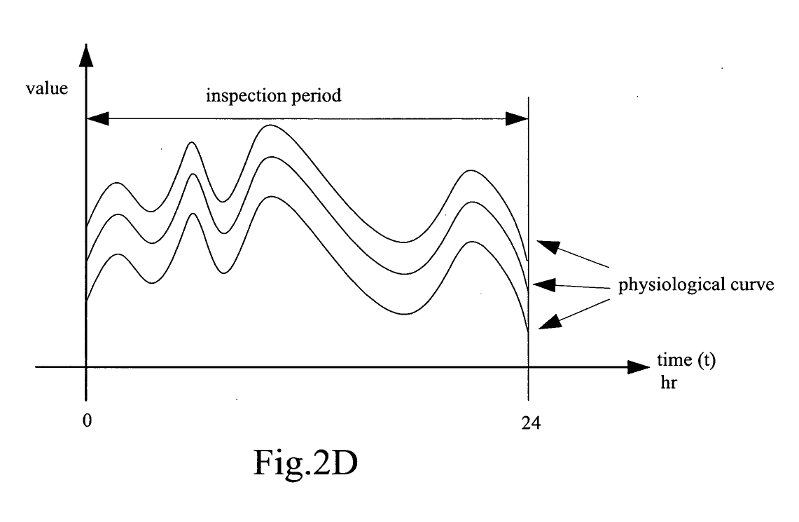

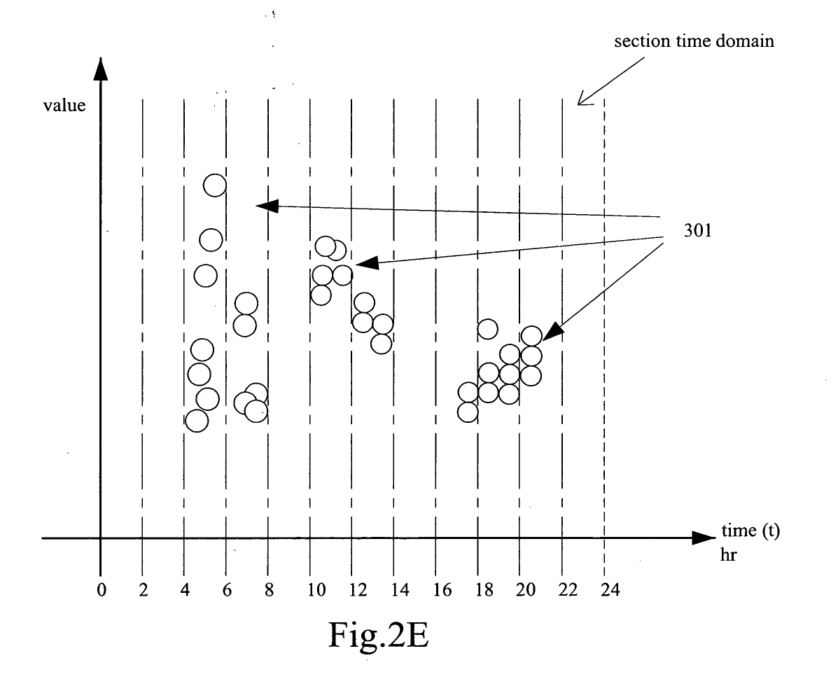

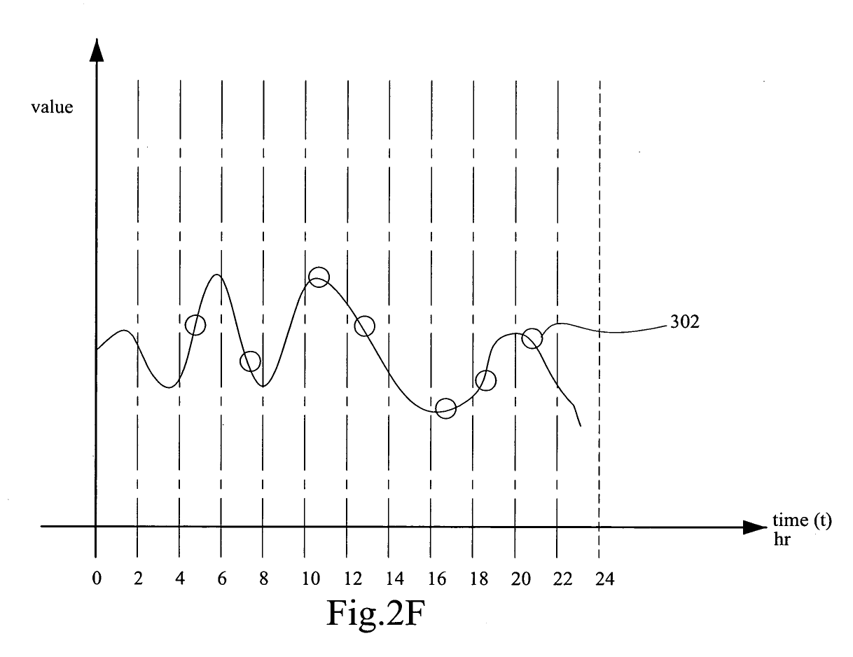

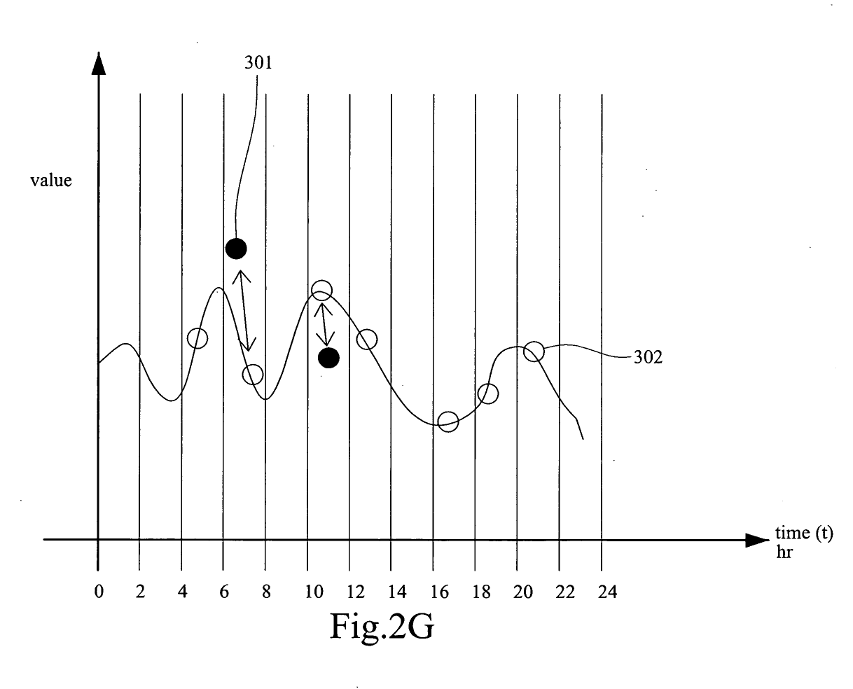

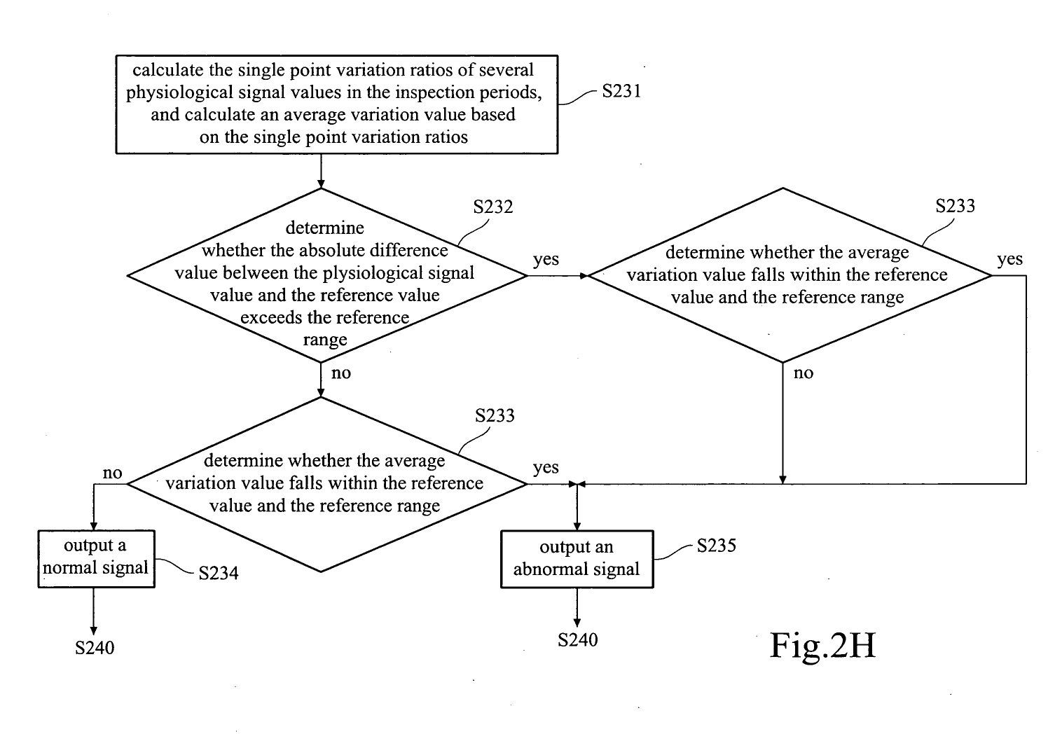

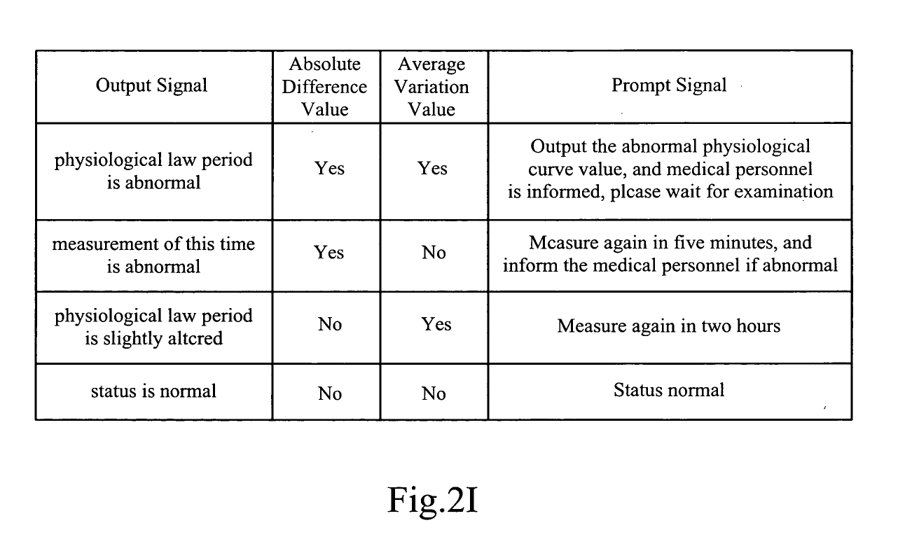

Referring to FIG. 2A, it is a flow chart of the method of the present invention, which can be understood together with FIG. 1. The method includes the following steps.

A plurality of specific physiological values is collected (Step S210), wherein the specific physiological values are constituted by all the physiological signal values inspected in the same specific section in a plurality of inspection periods.

First, the receiving module 102 receives a plurality of physiological signal values provided by the inspection device 110 in a time range, and stores the physiological signal values into the physiological database 103. The time range represents the time the inspection device 110 spent for continuously inspecting the physiological signal values.

Referring to FIG. 2B, it is a view of the collection of the physiological signal values of the present invention. The care device continuously receives a physiological signal value 301 via the receiving module 102 in a time range. The physiological signal value can be a blood pressure value, a heartbeat value, a blood sugar value, or a brainwave value.

For example, in continuous 30 days, the user utilizes the inspection device 110 to inspect the physiological signal value 301 three times a day. Thus, 30 days stands for the time range, and as the inspection is taken three times a day, the receiving module 102 receives ninety physiological signal values and stores all the physiological signal values into the physiological database 103.

Referring to FIG. 2C together, it is a schematic cut view of the time range. The data processing module 101 retrieves all the physiological signal values 301 from the physiological database 103, and divides the time range into a plurality of inspection periods with a time length as a unit.

In this embodiment, an inspection period is 24 hrs, i.e., a day. As the time range in which the user performs inspection continuously for 30 days, the data processing module 101 divides the time range into thirty inspection periods.

Together referring to FIGS. 2D and 2E, they are a schematic view of the inspection periods corresponding to the same time axis and a specific section view corresponding to the physiological signal values. The data processing module 101 generates a time axis, and all the inspection periods are corresponding to the time axis. The time axis has a plurality of specific sections of the same time length, and the total time lengths of all specific sections is equal to the time length of each inspection period.

Therefore, the physiological signal values of each inspection period are corresponding into the specific sections based on the measurement time, such that each specific section has physiological signal values of a different quantity.

In this embodiment, each specific section represents 2 hrs, so the time axis has twelve specific sections. As the user measures three times a day, each inspection period includes three physiological signal values 301. The three physiological signal values 301 are respectively corresponding into the twelve specific sections based on each measurement time. Moreover, the quantity of the inspection periods is thirty, so ninety physiological signal values 301 are respectively corresponding into the twelve specific sections.

However, each physiological signal value 301 has a different measurement time, so each specific section has a different quantity of physiological signal values. The data processing module 101 constitutes a plurality of specific physiological values according to the physiological signal values 301 contained in each specific section, and each specific section is corresponding to a specific physiological value.

A reference value and a reference range are calculated according to the specific physiological values (S220). The data processing module 101 calculates a reference value (Vi), time point (Ti), and reference range of the specific physiological values via two formulas Vi=(Σwi*M)/(Σwi) and Ti=(Σwi*ti)/(Σwi), and stores all the specific physiological values into the reference database 104.

i stands for the staging of the inspection periods, Mi stands for the physiological signal value of the specific section of the ith inspection period, and wi stands for a weight value of the ith inspection period. In addition, wi decreases along with the time interval between the ith inspection period and the next inspection period, wherein the longer the time interval is, the smaller the weight value will be.

Referring to FIG. 2F together, it is a curve diagram formed by specific physiological values. The reference value (Vi) and time point (Ti) are calculated by the above constituted specific physiological values 302. Then, a reference curve is formed by the specific sections corresponding to the specific physiological values 302.

It is determined whether the physiological signal value of the next inspection period falls within the reference value and the reference range according to the reference value and the reference range (S230).

Referring to FIG. 2G together, it is a schematic comparison view of the physiological signal values and the specific physiological values.

The care device utilizes the receiving module 102 to receive a physiological signal value measured by the user in a certain specific section in the next inspection period, and stores the physiological signal value into the physiological database 103 to be further transmitted to the data processing module 101.

The data processing module 101 selects specific physiological values with a time point (Ti) closest to the measurement time of the physiological signal value from the reference database 104, and determines whether the measured physiological signal value falls within the reference value and the reference range of the selected specific physiological values based on a comparison rule.

Referring to FIG. 2H, it is a flow chart of the comparison method, which includes the following steps.

The single point variation ratios of several physiological signal values in the inspection periods are calculated, and an average variation value is calculated based on the single point variation ratios (S231).

First, the data processing module 101 calculates the single point variation ratios of all physiological signal values in a specific period of time, and calculates an average variation value based on the single point variation ratios.

The formula of single point variation ratio is: physiological signal value (Mi)-reference value (Vi)|/reference value (Vi).

The specific period of time is a time length with the latest measurement time of the physiological signal value as a start point and calculating back for at least one inspection period.

It is determined whether the absolute difference value between the physiological signal value and the reference value exceeds the reference range (S232).

It is determined whether the average variation value falls within the reference value and the reference range (S233).

The data processing module 101 determines whether the physiological status of the user is normal or not based on the above two determination results. If the two comparison results are both negative, a normal signal is output (S234). On the contrary, if at least one of the two comparison results is “YES”, an abnormal signal is output (S235).

For example, according to FIG. 2G, the care device respectively obtains a physiological signal value from two specific sections of the 6th-8th hour and the 10th-12th hour.

Therefore, the data processing module obtains two specific physiological values with a time point closest to the two physiological signal values, so as to respectively determine whether the two physiological signal values are normal or abnormal, and output a normal result or abnormal signal according to the comparison result.

If the inspected physiological signal value 301 is a blood pressure contraction value, the blood pressure value measured in the specific section of the 6th-8th hour is 142 mmHg, and the reference value of the corresponding specific physiological value 302 is 120 mmHg, while the reference range thereof is 20 mmHg. The calculated absolute difference value between the physiological signal value 301 and the specific physiological value 302 is 22 mmHg which exceeds the reference range of 20 mmHg. Thus, the data processing module 101 outputs an abnormal signal, indicating that the measured physiological signal value 301 is an abnormal value.

Next, the blood pressure value measured in the specific section of the 10th-12th hour is 128 mmHg, and the reference value of the corresponding specific physiological value 302 is 140 mmHg, while the reference range thereof is 20 mmHg. The calculated absolute difference value between the physiological signal value 301 and the specific physiological value 302 is 12 mmHg, which does not exceed the reference range of 20 mmHg. Thus, the data processing module 101 outputs a normal signal, indicating that the measured physiological signal value 301 is a normal value.

Afterwards, the data processing module 101 calculates the single point variation ratios of the two physiological signal values 301, wherein one is 18.3%, the other is 8.5%, and the average variation value is 13.4%. The average variation value is compared with the pre-calculated reference range. If the pre-calculated reference range of the data processing module is 20%, the data processing module 101 determines the variation curve of the inspection period as a normal curve value, and outputs a normal signal. On the contrary, if pre-calculated reference range of the data processing module is 10%, an abnormal signal is output.

Finally, whether the physiological signal value of the next inspection period falls within the reference value and the reference range is displayed (S240).

The data processing module 101 utilizes the display unit to display the comparison result, i.e., displaying whether the measured physiological signal value exceeds the reference value and the reference range. If the comparison result is abnormal, an abnormal signal and a prompt signal are output. If the comparison result is normal, a normal signal is output, and the value of the specific physiological value (Ti, Vi) is updated.

The method of updating the specific physiological value (Ti, Vi) involve the following steps. The data processing module 101 retrieves all the specific physiological values in the specific section corresponding to the specific physiological value (Ti, Vi), substitutes the physiological signal value of the earliest inspection period with the physiological signal value of the latest specific section, re-calculates the value of the specific physiological value, and stores the re-calculated specific physiological value into the reference database 104.

Referring to FIG. 2I together, it is an output signal table of the data processing module 101, which takes the comparison result of whether the absolute difference value between the physiological signal value and the reference value exceeds the reference range, and the comparison result of whether the average variation value exceeds the reference range as rules.

When neither the difference absolute value nor the average variation value exceeds the reference range, a message of normal status is output.

When the difference absolute value does not exceed reference range, but the average variation value exceeds the reference range, a general abnormal signal indicating that the physiological law period is slightly altered is output, and meanwhile a prompt signal such as a message saying “measure again in two hours” is output.

When the difference absolute value exceeds the reference range, but the average variation value does not exceed the reference range, for example, a message saying “measure again in five minutes, and inform medical personnel if abnormal” is displayed.

When both the difference absolute value and the average variation value exceeds the reference range, a serious abnormal signal indicating that the physiological law period is abnormal is output, and meanwhile a prompt signal such as a message saying “output abnormal physiological curve value and the medical personnel is informed, please wait for examination” is output.

Therefore, in view of the above, the present invention can properly adjust the reference value automatically when performing measurement, so as to accurately inspect the physiological status based on the actual physiological status of the user, and prompt the user to rapidly solve the problems in physiological status.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A method of monitoring physiological status for periodically inspecting physiological status of a user, obtaining a physiological signal value, and determining whether the physiological signal value is normal or not, the method comprising:
collecting a plurality of specific physiological values, wherein the specific physiological values are constituted by the physiological signal values obtained in the same specific sections in a plurality of inspection periods;
calculating a reference value and a reference range according to the specific physiological values; and
determining whether the physiological signal value of the specific section of the next inspection period falls within the reference value and the reference range according to the reference value and the reference range.

2. The method of monitoring physiological status as claimed in claim 1, further comprising a step of displaying whether the physiological signal value of the specific section of next inspection period falls within the reference value and the reference range.

3. The method of monitoring physiological status as claimed in claim 1, further comprising:
calculating the single point variation ratios of several physiological signal values in the inspection periods, and calculating an average variation value based on the single point variation ratios; and
determining whether the average variation value falls within the reference value and the reference range.

4. The method of monitoring physiological status as claimed in claim 1, wherein the reference value is equal to (Σwi*Mi)/(Σwi), where i stands for the staging of the inspection periods, Mi stands for the physiological signal value of the specific section of the iₜₕ inspection period, and wi stands for a weight value of the iₜₕ inspection period; wi decreases along with the time interval between the iₜₕ inspection period and the next inspection period, and the longer the time interval is, the smaller the weight value is.

5. A care device of monitoring physiological status, connected to an inspection device for providing at least one physiological signal value, wherein the care device periodically inspects physiological status of a user, obtains a physiological signal value, and determines whether the physiological signal value is normal or not, and the care device comprises:
a physiological database;
a reference database;
a receiving module, electrically connected to the inspection device and the physiological database, for receiving and storing the physiological signal values into the physiological database; and
a data processing module, electrically connected to the physiological database, the reference database, and the receiving module, for retrieving the physiological signal values of the same specific section from the physiological database according to the plurality of inspection periods corresponding to the physiological signal values, so as to calculate a reference value and a reference range of the plurality of specific physiological values, and store the specific physiological values into the reference database, wherein the data processing module determines whether the physiological signal value of the specific section of the next inspection period falls within the reference value and the reference range according to the reference value and the reference range, and determines whether or not to update the specific physiological values stored in the reference database according to the comparison result.

6. The care device of monitoring physiological status as claimed in claim 5, further comprising a display unit, electrically connected to the data processing module, for displaying whether the physiological signal value of the next inspection period falls within the reference value and the reference range.

7. The care device of monitoring physiological status as claimed in claim 6, wherein the display unit is a liquid crystal panel.

8. The care device of monitoring physiological status as claimed in claim 5, further comprising a sound playing unit electrically connected to the data processing module, wherein the data processing module outputs an auxiliary sound signal when the comparison result is generated, and the sound playing unit receives the auxiliary sound signal and plays a sound corresponding to the auxiliary sound signal.

9. The care device of monitoring physiological status as claimed in claim 5, further comprising a signal transmission module electrically connected to the data processing module and the receiving module, and connected to at least one network device, such that the network device is electrically connected to the data processing module and the receiving module via the signal transmission module, thus obtaining the physiological signal values or the reference value and reference range of the specific physiological values.

10. A record medium for monitoring physiological status, the medium having computer-readable program code for periodically inspecting physiological status of a user, obtaining a physiological signal value, and determining whether the physiological signal value is normal or not, the record medium executing the steps comprising:
collecting a plurality of specific physiological values, wherein the specific physiological values are constituted by the physiological signal values obtained in the same specific sections in a plurality of inspection periods;
calculating a reference value and a reference range according to the specific physiological values; and
determining whether the physiological signal value of the specific section of the next inspection period falls within the reference value and the reference range according to the reference value and the reference range.

11. The record medium of monitoring physiological status as claimed in claim 10, wherein the record medium further executes a step of displaying whether the physiological signal value of the specific section of next inspection period falls within the reference value and the reference range.

12. The record medium of monitoring physiological status as claimed in claim 10, wherein the record medium further executes steps comprising:
calculating the single point variation ratios of several physiological signal values in the inspection periods, and calculating an average variation value based on the single point variation ratios; and
determining whether the average variation value falls within the reference value and the reference range.

13. The record medium of monitoring physiological status as claimed in claim 10, wherein the reference value is equal to (Σwi*Mi)/(Σwi), where i stands for the staging of the inspection periods, Mi stands for the physiological signal value of the specific section of the iₜₕ inspection period, and wi stands for a weight value of the iₜₕ inspection period; wi decreases along with the time interval between the iₜₕ inspection period and the next inspection period, and the longer the time interval is, the smaller the weight value is.
